(19) United States
(12) Patent Application Publication
(10) Pub. No.: US 2021/0339004 A1
(43) Pub. Date: Nov. 4, 2021

(54) LINE DEVICE FOR A VENTRICULAR ASSIST DEVICE AND METHOD FOR PRODUCING A LINE DEVICE

(71) Applicant: KARDION GMBH, Stuttgart (DE)

(72) Inventors: Thomas Alexander Schlebusch, Renningen (DE); David Minzenmay, Stuttgart (DE); Julian Kassel, Böblingen (DE); Tobias Baechle, Stuttgart (DE)

(21) Appl. No.: 15/734,331

(22) PCT Filed: Jun. 6, 2019
(86) PCT No.: PCT/EP2019/064775

§ 371 Date: Jun. 14, 2021

(30) Foreign Application Priority Data
Jun. 6, 2018 (DE) 10 2018 208 911.6

(51) Int. Cl. A61M 60/857; A61M 60/139; A61M 60/17

(52) U.S. Cl. CPC A61M 60/857; A61M 60/139; A61M 60/17; A61M 2207/00; A61M 2205/0238; A61M 2205/04; A61M 2205/0233

(57) ABSTRACT

The invention relates to a line device (105) for a ventricular assist system (100), wherein the line device (105) comprises a guide cannula (145), which is structured at least partially along a direction of extent; and, furthermore, the line device (105) comprises an electrical conducting element (145), which is arranged in the guide cannula (140), wherein the electrical conducting element (145) comprises a multilayer structure.

The invention is based on a line device or a method, as defined in the preamble of the independent claims. The subject matter of the present invention is also a computer program.

In the meantime significant advancements in the material sciences have made it possible to produce electrical conductor structures that are thin, flexible and simultaneously complexly structured, as, for example, the publication by Burkard et al.: Flex Technology for Foldable Medical Flip Chip Devices; IMAPS Conf. on Device Packaging, Scottdale Ariz., Mar. 17-20, 2008, describes. In the field of medical technology such electrical conductor structures are used, for example, in the form of implanted intraocular pressure sensors or retina implants.

Based on the aforesaid, the object of the present invention is to provide a line device, which is intended for a ventricular assist system and which is simplified and improved in terms of its integration and functionality, and to provide an advantageous method for the production thereof.

Against this background, the approach, presented herein, can be used to provide a line device for a ventricular assist system; a method for producing a line device; furthermore, a device that uses said method; and finally a corresponding computer program in accordance with the main claims. The measures, listed in the dependent claims, make possible the advantageous further developments and improvements of the device, disclosed in the independent claim.

The line device that is presented herein and that is intended for a ventricular assist system describes a high frequency compatible, electrical conducting element, for example, based on a flexible substrate. Said electrical conducting element can integrate the functions of a sensor carrier, an electrical connecting line and a connection element in a single subassembly, so that, for example, it is possible to dispense with additional connecting points on the pump of the ventricular assist system. As a result, the production process can be simplified; and the reliability of the ventricular assist system can be increased.

A line device for a ventricular assist system is presented, wherein the line device comprises the following features:

a guide cannula that is structured at least partially along a direction of extent; and

an electrical conducting element that is arranged in, on or at the guide cannula, wherein the electrical conducting element comprises a multilayer structure.

A line device may be a component of a ventricular assist system, said line device being used to integrate a high frequency compatible electrical conducting element, for example, inside a guide cannula of the line device. A ventricular assist system, also called an artificial heart or a VAD (ventricular assist device), can be understood to mean a pump device for increasing the pumping capacity of a heart. The ventricular assist system can be inserted into a ventricle or the aorta by means of, for example, a catheter. In particular, the ventricular assist system can be a left ventricular assist system, which, for example, can also be designed as a percutaneous assist system, but does not have to be. A guide cannula may be a cylindrical housing, which can have, for example, a metal-containing alloy and/or a constant outer diameter, but alternatively can also exhibit a tapering. Therefore, the guide cannula can be used to receive an electrical conducting element or, more specifically, an electrical connecting line and can be used, for example, in a line device of a ventricular assist system. Furthermore, the guide cannula can also have a structured surface or structures in a sheath, which can be formed, for example, as a braid and/or as a spiral or wave structure, cut out of a tube, or as a serrated structure or as a zigzag variant. An electrical conducting element can be understood to mean an electrical connecting line, which is arranged, for example, inside a guide cannula of a ventricular assist system, and said electrical connecting line is used to make an electrical connection between a sensor system, for example, a pressure and/or temperature sensor, in a distal tip of the ventricular assist system and an electrical connecting cable at a proximal end of said ventricular assist system. A multilayer structure may be a multilayer structural construction of an electrical conducting element and/or an electrical connecting cable, wherein each individual layer can offer a specific functionality, for example, a conductive and/or an insulating function. Therefore, the multilayer structure can be produced, for example, by means of a thin film process.

The advantages of the approach that is presented herein and that is intended for a line device for a ventricular assist system are, for example, that an electrical conducting system of the line device is implemented, in particular, by using a thin film process, where in this case the thin film process offers a reduction in the thickness of the coat applied, as compared to a standard electrical connecting cable. Furthermore, the electrical conducting element can also be implemented, for example, in one piece and, in addition, can combine, for example, the functions of a sensor carrier, an electrical connecting line and a connection element in a single subassembly, with the result that this form of implementation reduces any possible fault points; and, moreover, any unnecessary contact points, which would represent an additional increase in the thickness of the coat applied, are eliminated. In order to avoid contact points, it is also proposed, for example, that a single (for example, one piece) flexible substrate be used not only in the sensor head unit as a guide of the electrical conducting element along the guide cannula but also for making electrical contact with a feed-through element on the end unit of the ventricular assist system. Said flexible substrate, for example, a thin film substrate, can be pre-fixed by means of an adhesive and subsequently coated with a protective lacquer layer that provides protection for the line device against possible damage.

In accordance with one embodiment, the electrical conducting element can comprise a plurality of (for example, coplanar) layers made of a conductive and/or insulating material, in particular, wherein a conductive layer comprises at least partially a gold material; and/or an insulating layer is made at least partially of a polyimide material. Such an embodiment of the approach, presented herein, offers the advantage that the combination of different layers for purposes of constructing an electrical conducting element can produce, for example, new and/or improved properties and fields of application of the electrical conducting element. Thus, the electrical conducting element can be fabricated, for example, using a thin film process, where in this case an implementation of the electrical conducting element by the thin film process offers the advantage of a reduction in the thickness of the coat applied. Furthermore, the production of such layers, for example, by means of wafer-based lithography processes makes it possible to achieve production processes of a line device that are both resource and energy efficient. The layers are produced, for example, by lithography (in particular, by applying the photoresist, exposure, development, base layer sputtering, galvanically thickening, photoresist removal).

Polyimide materials are used in electrical engineering, for example, on account of their heat resistance, low outgassing, radiation resistance and insulating properties in the form of light-brown, semi-transparent films. At the same time high continuous use temperatures of up to 230° C. and for a short time up to 400° C. are possible. Polyimide materials can be used, for example, in particular, for a particularly thin and, nevertheless, quite stable lacquer insulation of electrical lines in the thin film process. The multilayer construction of a conductor on, for example, a glass carrier substrate based on polyimide is particularly advantageous in that polyimide can be applied in liquid form by means of spin coating. In contrast to polyimide layers laminated with the aid of an adhesive (as is customary in the flexible printed circuit card industry), the production of insulating layers in a liquid manner makes it possible to hermetically enclose the metallic conductor, so that no moisture can enter; and corrosion problems are reduced. In the field of medical technology, polyimide is preferred due to its biocompatibility. A gold material offers the advantage that it does not form an oxide layer; and, as a result, good electrical contact is always ensured. The excellent biocompatibility should be underscored, in particular. Other conceivable metals are platinum-iridium or, in principle, also copper owing to its high conductivity and the low price.

In accordance with one embodiment, the electrical conducting element can comprise a shielding element, in particular, wherein the shielding element is implemented using the conductive layers and/or a through-contact between the individual layers. In this case the shielding can be produced, for example, by means of metallic layers and flat through-contacts between the individual layers of the electrical conducting element. Such an embodiment of the approach, presented herein, offers the advantage that the shielding can offer an improvement in the high frequency properties (for example, with respect to an impedance control) of the line device.

In accordance with one embodiment, the electrical conducting element can comprise a plurality of lines, wherein some of the lines are arranged inside a layer, in particular, wherein the majority of the lines are arranged outside the shielding element. Such an embodiment of the approach, presented herein, offers the advantage that the shielding element, which can comprise, for example, a metallic material, can be formed very simply in the contacting region of the electrical conducting element by means of processes that are also used for the production of conductor tracks or lines in the conducting element.

In accordance with one embodiment, the electrical conducting element can comprise a sensor contact region for contacting at least one sensor and/or a signal generator contacting region for contacting at least one signal generator. In this case the at least one sensor can be, for example, a temperature sensor, which measures the temperature of the blood of a patient suffering from a heart disease and/or a (for example, barometric) pressure sensor for detecting the ventricular pressure of a cardiac patient. The signal generator can be, for example, an ultrasonic element that allows a volume flow of the blood of a cardiac patient to be measured. Such an embodiment of the approach, presented herein, offers the advantage that such an implementation of one end of the electrical conducting element can be used to allow contact to be made with both the sensors and also the ultrasonic element.

In accordance with one embodiment, the sensor contact region can be designed to receive and/or to contact at least two sensors; and/or the sensor contact region can be formed in a rectangular manner, in particular, wherein the sensor contact region comprises at least two edges, wherein the sensor contact region is bent at the at least two edges. Such an embodiment of the approach, presented herein, offers the advantage that the sensor contact region is bent at the two edges, in order to wrap around a groove in the sensor head unit of the ventricular assist system and, in so doing, to ensure in this way a stable and permanent hold. This groove or, more specifically, the sensor cavity can be filled (for example, after embedding a sensor in this groove) with a potting compound, for example, a solid and/or gel-like silicone, for purposes of protecting the sensors from blood and mechanical damage. In a particular embodiment the straight regions between the bending edges can be reinforced by stiffening elements, so that bending is possible only in the region of the bending edge.

In accordance with one embodiment, the signal generator contacting region can comprise at least two bent contact points. The signal generator contacting region is designed, for example, as a circular printed circuit board, where in this case the at least two bent contact points are designed to receive and/or to contact at least one signal generator, for example, an ultrasonic element. Furthermore, the signal generator contacting region also comprises an edge. Such an embodiment of the approach, presented herein, offers the advantage that the signal generator contacting region can also be bent at the edge, in order to integrate itself in the cylindrical shape of the ventricular assist system in the best possible way.

In accordance with one embodiment, the electrical conducting element can comprise a connection point element, wherein the connection point element is shaped in a circular, hexagonal, square, triangular, generally polygonal shaped or U shaped manner, in particular, wherein the connection point element comprises a plurality of round connecting points and/or connecting points, arranged radially and/or circumferentially, on an external environment of the connection element. Such an embodiment of the approach, presented herein, offers the advantage that the semicircularly shaped connecting points can be folded over the thin and/or flexible lines of the electrical conducting element between the similarly radially and/or circumferentially arranged contact pins of a feed-through element, in order to be electrically contacted there by welding, conductive adhesive bonding or soldering. The shape of the connection point element as a circle with semicircular connecting points allows convenient contacting, so that even a small adjustment of the length can be implemented owing to the realization of said shape.

In accordance with one embodiment, a structured sheath of the guide cannula can be formed as a braid and/or as a spiral/wave or zigzag structure, cut out of a tube, in particular, wherein the guide cannula comprises a metal-containing alloy. Such an embodiment of the approach, presented herein, offers the advantage that the electrical conducting element is mechanically protected and/or supported by means of a braided and/or spirally shaped structure of the guide cannula. If the cable or, more specifically, the conducting element is integrated in a braided tube without any other devices, then high flexural loads act on the cable, an aspect that can lead to a break in the electrical connection before the time horizon of a permanent implant. Therefore, it is advantageous to preassemble the cable on a supporting or protective structure, for example, a metallic strip, and to integrate the latter in the braid in the following. If the guide cannula is cut out of a tube, then the shape of a supporting or protective structure can be integrated in the cutting program, so that a separate component is not required. In particular, a guide of the electrical conducting element over a spirally shaped web of a guide cannula offers the electrical conducting element extremely high protection against a mechanical flexural load over long periods.

In accordance with one embodiment, each layer of the electrical conducting element can have a thickness in a range between 5 μm and 15 μm; and/or the electrical conductor can be formed in a meandering shape. Each layer of polyimide (PI) or gold can be, for example, about 5 to 15 μm thick. Then the total thickness of the electrical conducting element is a function of the number of layers and the thickness of the individual layers, where in this case the number of layers is also dependent on an existing shielding or, more specifically, shielding layer. For example, a 3 layer system (PI, gold, PI) can have a maximum thickness of 15 μm. For example, such a system with shielding can have a maximum thickness of 11 layers at a maximum of 10 μm each, i.e., 110 μm. Such an embodiment of the approach, presented herein, offers the advantage that in the event of an expansion or compression an adjustment of the length of the conducting element can be achieved by means of an electrical conducting element that is formed in a meandering shape.

Furthermore, the approach, presented herein, provides a ventricular assist system having a line device in accordance with a variant, presented herein, wherein the line device is arranged between a sensor head unit and an end unit of the ventricular assist system, in particular, wherein one connecting element each is arranged between the line device and the sensor head unit and/or between the line device and the end unit. The particular advantages of the approach, presented herein, can also be realized in a simple and cost effective manner by means of such an embodiment.

Finally, a method for producing a line device is presented, wherein the method comprises the following steps of:
providing the guide cannula and the electrical conducting element; and
arranging the electrical conducting element inside the guide cannula, in order to produce the line device.

The method, which is presented herein, for producing a line device for a ventricular assist system can be implemented, for example, in software or hardware or in a mixed form of software and hardware, for example, in a control device.

Furthermore, the approach, presented herein, also provides a device that is designed to execute, trigger or, more specifically, implement the steps of a variant of the method, presented herein, and that is intended for producing a line device for a ventricular assist system in corresponding apparatuses. The problem, on which the invention is based, can also be solved quickly and efficiently by means of this alternative variant of the invention in the form of a device.

For this purpose the device can comprise at least one computing unit for processing signals or data, at least one memory unit for storing signals or data, at least one interface to a sensor or an actuator for reading in sensor signals from the sensor or for outputting data or control signals to the actuator and/or at least one communication interface for reading in or outputting data that are embedded in a communication protocol. The computing unit can be, for example, a signal processor, a microcontroller or the like; and the memory unit can be a flash memory, an EEPROM or a magnetic memory unit. The communication interface can be designed to read in or output data in a wireless and/or wired manner, where in this case a communication interface, which can read in or output data in a wired manner, can read in, for example, electrically or optically said data from a corresponding data transmission line or can output said data into a corresponding data transmission line.

A device in the present case can be understood to mean an electrical device, which processes sensor signals and, as a function thereof, outputs control signals and/or data signals. The device can comprise an interface that can be configured in hardware and/or software. In the case of a design in hardware, the interfaces can be, for example, part of a so-called ASIC system, which includes a wide range of functions of the device. However, it is also possible for the interfaces to be separate, integrated circuits or to consist at least partially of discrete components. In the case of a design in software, the interfaces can be software modules that are present, for example, on a microcontroller, in addition to other software modules.

Advantageous is also a computer program product or computer program having program code that can be stored on a machine-readable carrier or storage medium, such as a semiconductor memory, a hard disk memory or an optical memory and is used to execute, implement and/or trigger the steps of the method in accordance with any one of the embodiments described above, in particular, if the program product or program is executed on a computer or a device.

Exemplary embodiments of the approach, presented herein, are shown in the drawings and explained in more detail in the following description. The drawings show in:

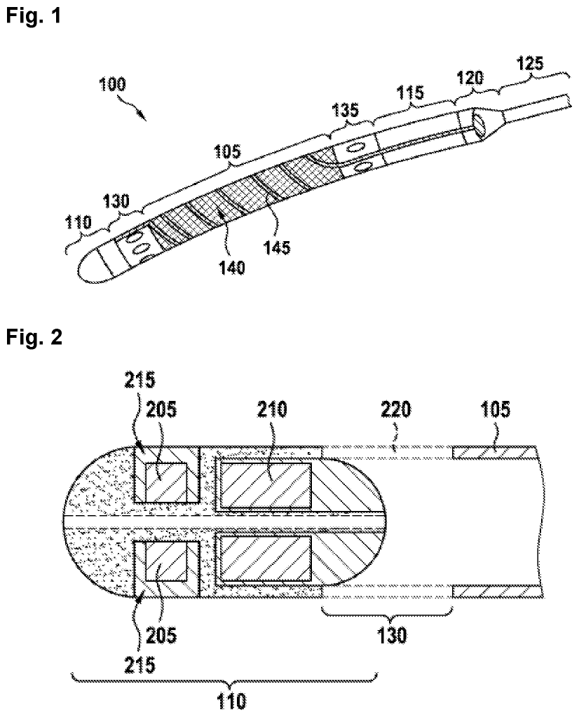

FIG. 1 a schematic view of a left ventricular assist system with an integrated line device in accordance with one exemplary embodiment;

FIG. 2 a schematic view of a sensor head unit of a left ventricular assist system in accordance with one exemplary embodiment;

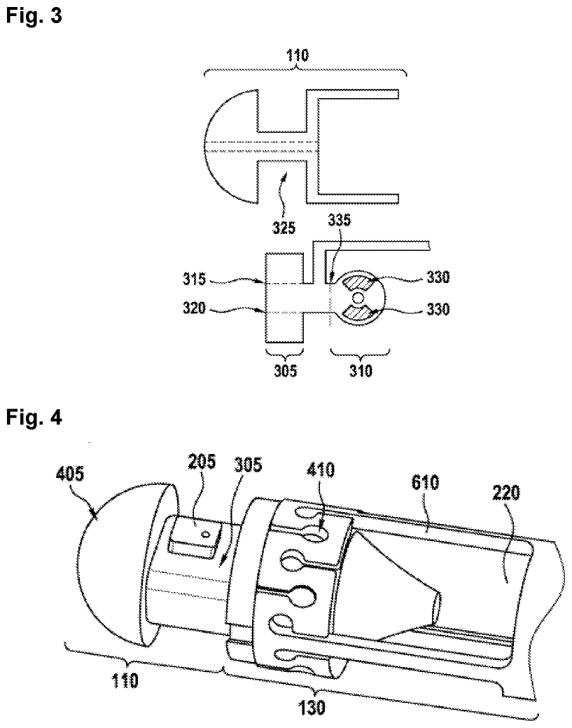

FIG. 3 a schematic view of a sensor contact region and a signal generator contacting region of an electrical conducting element in accordance with one exemplary embodiment;

FIG. 4 a three dimensional view of a sensor head unit of a left ventricular assist system in accordance with one exemplary embodiment;

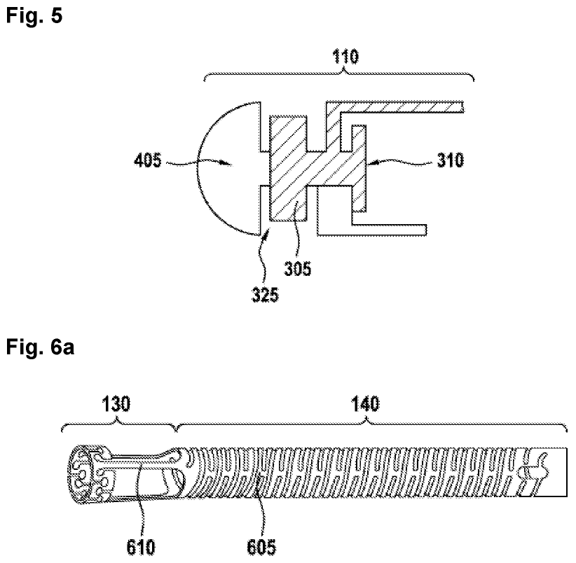

FIG. 5 a schematic view of a sensor head unit of a left ventricular assist system in accordance with one exemplary embodiment;

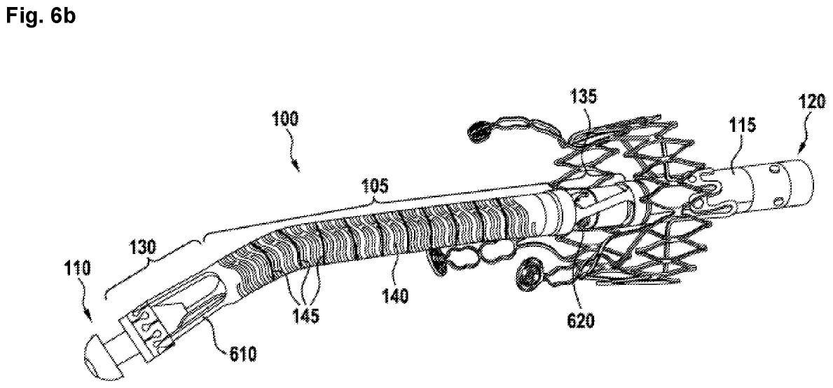

FIG. 6 a schematic view of a guide cannula of a line device in accordance with one exemplary embodiment;

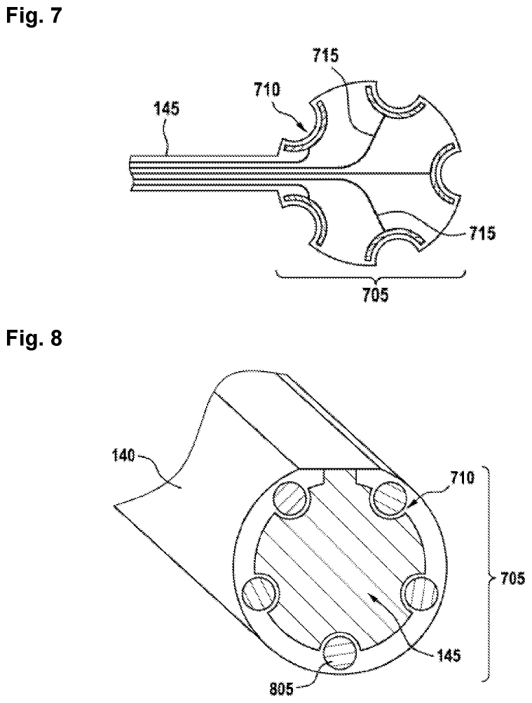

FIG. 7 a schematic view of a connection point element of an electrical conducting element in accordance with one exemplary embodiment;

FIG. 8 a schematic view of a contacted connection point element of an electrical conducting element in accordance with one exemplary embodiment;

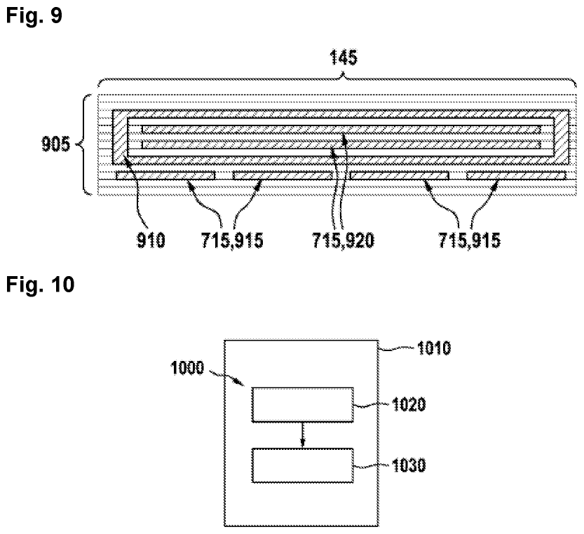

FIG. 9 a schematic cross sectional view of an electrical conducting element in accordance with one exemplary embodiment; and

FIG. 10 a flowchart of an exemplary embodiment of a method for producing a line device in accordance with one exemplary embodiment.

In the following description of advantageous exemplary embodiments of the present invention, identical or similar reference numerals are used for those elements that are shown in the various figures and that act in a similar manner, thus dispensing with a repeated description of these elements.

FIG. 1 shows a schematic view of a left ventricular assist system 100 with an integrated line device 105 in accordance with one exemplary embodiment. The ventricular assist system 100 comprises a cylindrically shaped, elongated structure with a substantially constant outer diameter and rounded, tapering ends for ease of placement by means of catheters in a blood vessel, such as the left ventricle or the aorta.

To begin with, the ventricular assist system 100 (here by way of example a left ventricular assist system 100 for percutaneous implantation into a left ventricle) comprises the line device 105, where in this case the line device 105 is arranged between a sensor head unit 110 and a motor housing 115, an end unit 120 and a connecting cable 125 of the ventricular assist system 100. In this respect the line device can be connected to the sensor head unit 110 and the motor housing 115 or, more specifically, the end unit 120 by means of one connecting element 130 and 135 each. The connecting elements 130 and 135 contain openings for receiving or discharging the blood. The coupling is effected, for example, by adhesive bonding. The line device 105 and the connecting element 130 can also consist of one part. That means that they can be manufactured in one piece. The sensor head unit 110 and the connecting element 130 in one embodiment can also be made of one part, i.e., in one piece.

The sensor head unit 110 of the ventricular assist system 100 comprises, for example, a tip in the form of a sensor assembly that is used, for example, for measuring the pressure and/or the temperature. The end unit 120 represents, for example, a proximal end of the ventricular assist system 100 and forms a transition between the motor housing 115 of the ventricular assist system 100 and the connecting cable 125 for connecting the ventricular assist system 100 to an external energy source or an external evaluating device or control device.

The line device 105 comprises a guide cannula 140 that comprises, at least partially along a direction of extent, a structure or, more specifically, a surface that is structured here. For example, the guide cannula 140 comprises a spiral-shaped surface structure. An electrical conducting element 145 is arranged inside the guide cannula 140, where in this case said electrical conducting element 145 is used for the electrical connection of the sensor head unit 110 to the connecting cable 125 at the proximal end of the ventricular assist system 100.

In accordance with one exemplary embodiment, the electrical conducting element 145 can contain a meander, in order to achieve a length adjustment of the same. In this case the meander is placed preferably in the region of the motor housing 115.

FIG. 2 shows a schematic view of a sensor head unit 110 of a left ventricular assist system 100 in accordance with one exemplary embodiment.

The sensor head unit 110 of the ventricular assist system 100 comprises, for example, a tip in the form of a sensor assembly that is used, for example, for measuring the pressure and/or the temperature of a patient suffering from a heart disease. For this purpose the sensor head unit 110 in accordance with one exemplary embodiment comprises two sensors 205 and a signal generator 210. The two sensors 205 may be, for example, a pressure sensor and/or a temperature sensor. The signal generator 210 can be, for example, an ultrasonic element. In accordance with one embodiment, both sensors 205 are arranged in a sensor cavity 215, which is filled with a potting compound for protecting the sensors 205 from blood and/or mechanical damage. Thus, this potting compound can be, for example, a solid and/or gel-like silicone and/or a silicone oil.

As shown in the schematic view of a sensor head unit 110 illustrated in this embodiment, the sensor head unit 110 is connected to the line device 105 by means of the connecting element 130, where in this case the connecting element 130 comprises a plurality of inlet windows 220, through which the blood of the cardiac patient enters the ventricular assist system.

FIG. 3 shows a schematic view of a sensor contact region 305 and a signal generator contacting region 310 of an electrical conducting element in accordance with one exemplary embodiment.

In accordance with one exemplary embodiment, the electrical conducting element comprises, for example, a structure on at least one of its ends; said structure serves as a sensor contact region 305 for directly mounting and/or contacting at least one sensor and/or as a signal generator contacting region 310 for contacting at least one signal generator by conductive adhesive bonding, soldering and/or bonding. In this case the sensor contact region 305 and the signal generator contacting region 310 are arranged on the sensor head unit 110, which is used, for example, to measure the pressure and/or the temperature of a cardiac patient. The sensor contact region 305 is designed, for example, as a rectangular printed circuit board, in order to receive and/or to contact at least two sensors. Furthermore, the sensor contact region comprises two edges 315 and 320, where in this case the sensor contact region 305 can be bent at these two edges 315 and 320, in order to wrap around a groove 325 in the sensor head unit 310. The signal generator contacting region 310 is designed, for example, as a circular printed circuit board, in order to receive and/or to contact at least one signal generator, for example, an ultrasonic element. For this purpose the signal generator contacting region 310 in accordance with one exemplary embodiment comprises two bent contact points 330, in order to contact the ultrasonic element. Furthermore, the signal generator contacting region 310 also comprises an edge 335, on which it can be bent, in order to integrate itself in the cylindrical shape of the ventricular assist system in the best possible way.

FIG. 4 shows a three dimensional view of a sensor head unit 110 of a left ventricular assist system in accordance with one exemplary embodiment. The sensor head unit 110 comprises a sensor head 405, which is formed, by way of example, in a mushroom shaped manner; furthermore, the sensor contact region 305, on which a sensor 205 is mounted; and finally the connecting element 130, which comprises a plurality of inlet windows 220, through which the blood of the cardiac patient enters the ventricular assist system. Furthermore, a joining region 410 is provided that is used to press fit the connecting element 130 to the sensor head unit 110. The inlet windows 220 are defined by three webs 610, two of which are visible on the right side in FIG. 4. In order to minimize a possible pressure loss, the inlet windows are designed to be as large as possible, so that the thin webs 610 remain in the region 130. As shown in the schematic view of a sensor head unit 110 illustrated in this embodiment, the sensor head unit 110 is connected in a fluid tight manner to the line device 105 by means of the connecting element 130.

FIG. 5 shows a schematic view of a sensor head unit 110 of a left ventricular assist system in accordance with one exemplary embodiment. In accordance with one exemplary embodiment, the sensor head unit 110 comprises a sensor head 405, which is formed, by way of example, in a mushroom shaped manner; furthermore, the sensor contact region 305, on which at least one sensor is mounted and/or contacted; and, furthermore, the signal generator contacting region 310, on which at least one signal generator is contacted. A fitting of the sensor contact region 305 and/or of the signal generator contacting region of the electrical conducting element into the sensor head unit 110 is shown in the schematic view of the sensor head unit 110 illustrated in this embodiment, where in this case the sensor contact region 305 is bent at its at least two edges, in order to wrap around the groove 325 of the sensor head unit 110. Furthermore, the signal generator contacting region 310 is also bent at its edge, in order to integrate itself in the cylindrical shape of the ventricular assist system in the best possible way.

FIG. 6 shows a schematic view of a guide cannula 140 of a line device in accordance with one exemplary embodiment.

In accordance with one exemplary embodiment, the inlet cannula 140 is formed as a type of flexible cylindrical feed tube with a continuous, structured surface 605 for guiding the electrical conducting element. In this case the flexible guide cannula 140 is designed, for example, as a structure, which is cut out of a tube and which comprises a constant outer diameter, where in this case the cut pattern contains a continuous spiral 605 for supporting and protecting the electrical conducting element. Furthermore, the guide cannula comprises, for example, an integrated connecting element 130 consisting of a joining region 410 and a plurality of webs 610 as a transition region, with which the guide cannula 140 is integrally connected to the connecting element 130. In this respect the connecting element 130 is made, for example, of the same material as a metallic alloy. The region of the connecting element 130 between the joining region 410 and the guide cannula forms the inlet windows, which are separated from one another by thin webs 610 (to which the electrical conducting element 145 can be guided) and through which the blood of the cardiac patient enters the ventricular assist system.

In an alternative exemplary embodiment of the guide cannula 140, the latter is formed as a braid, in which a flat strip is embedded, for example, as a support of the electrical conducting element, for the purpose of protecting the electrical conducting element. Said flat strip can also comprise a metallic alloy, for example, a nickel-titanium alloy.

FIG. 6B shows a schematic representation of a ventricular assist system 100 with a line device 105. The sensor head 110 and the guide cannula 140, which is made, for example, of a NiTiNol material and which comprises the connecting element 130 and the line device 105, can also be seen in this embodiment. At the front in the region of the connecting element 130, the blood runs between the webs 610 into the ventricular assist system 100 in the state inserted in the patient. For example, for manufacturing reasons, the connecting element 135 is designed here as an individual part. The motor housing 115 and the end unit 120 (which, for example, can contain contact pins, which are not shown in FIG. 6B and which are intended for contacting a cable through the body of the patient) are hermetically welded to one another or, more specifically, are connected to one another in a fluid tight manner at the rear of the motor housing 112. The electrical conducting element 145 is guided on the outside of the device or, more specifically, the guide cannula 140 from the tip of the sensor head 110 over the webs 610 of the inlet cage or, more specifically, the connecting element 130 and the spiral as part of the guide cannula 140 to the squirrel cage or, more specifically, to the additional connecting element 135, there over another web 620, which corresponds to the web 610, then through the motor or, more specifically, the motor housing 115 as far as to the end unit 120, at which the electrical conducting element 145 can then be electrically contacted with the line 125.

FIG. 7 shows a schematic view of a connection point element 705 of an electrical conducting element 145 in accordance with one exemplary embodiment.

In accordance with one exemplary embodiment, the electrical conducting element 145 comprises the connection point element 705 on one of its ends. In this case the connection point element 705 is formed, as an example, in a circular shape. However, in an alternative exemplary embodiment it can also be formed in an O-shaped or U-shaped, hexagonal, square, triangular or generally polygonal shaped manner. Furthermore, in accordance with one exemplary embodiment, the connection point element 705 comprises a plurality of connecting points 710, which are arranged radially and/or circumferentially on an external environment of the connection point element 705. In this case the connecting points 710 are designed so as to be round or semicircular, so that a plurality of thin and flexible lines 715 of the electrical conducting element 145 can be folded between the similarly radially arranged contact pins of a feed-through element (not shown), in order to be electrically contacted there by means of welding, conductive adhesive bonding and/or soldering.

FIG. 8 shows a schematic view of a contacted connection point element 705 of an electrical conducting element 145 in accordance with one exemplary embodiment at the proximal end 120 of a ventricular assist system. In accordance with one exemplary embodiment, the connection point element 705, which is depicted, is a fully contacted connection point of the electrical conducting element 145. In this view the multilayer structure of the electrical conducting element 145 is clearly visible. The connection point element 705 comprises a plurality of semicircular connecting points 710, which are arranged radially and/or circumferentially on an external environment of the connection point element 705. In this case each connecting point 710 is connected to one contact pin 805 each of a feed-through element (not shown) at the proximal end 120 of the support system, where in this case the feed-through element is also a conducting element that is used to connect, for example, the power supply lines of the electric motor 115 to the supply cable 125. In this case the contacting of the electrical conducting element 145 initially at the metallic pins of the back end 120 enables a robust mechanical coupling as a common connecting element of the flexible conductors of the connecting cable 125 and of the electrical conducting element. A direct connection of the conductors of the connecting cable to the electrical conducting element is not to be recommended mechanically.

FIG. 9 shows a schematic cross-sectional view of an electrical conducting element 145 in accordance with one exemplary embodiment.

In accordance with one exemplary embodiment, the electrical conducting element 145 comprises a plurality of coplanar layers 905 made of a conductive and/or insulating material. In this case the conductive layers comprise, by way of example, at least partially a gold material; and the insulating layers are made at least partially of a polyimide material. Furthermore, the electrical conducting element 145 also comprises a shielding element 910, which is implemented, for example, using the conductive layers and is based on a gold material. The shielding element 910 has, for example, a width of 470 μm and a thickness of 10 μm. As an alternative to the exemplary embodiment of a shielding element 910 shown in this embodiment, the shielding element 910 can also be implemented using a through-contacting between the individual layers. In this case the shielding element 910 is used to shield individual conductors or conductor pairs of the electrical connecting element, in order to prevent the occurrence of any electrical and/or magnetic coupling of fields into the lines of the electrical connecting element, in particular, at higher frequencies, or conversely to reduce the electromagnetic radiation from the electrical connecting element. In addition, the shielding can be used to adjust a specific wave resistance of the electrical connecting element and to reduce the influence of the environment for reasons of high frequency compatibility.

Furthermore, the electrical conducting element 145 comprises a plurality of lines 715, where in this case said lines 715 are arranged inside a layer and have, for example, a width of 410 μm and a thickness of 10 μm. Thus, in accordance with one exemplary embodiment, the electrical conducting element 145 comprises four digital lines 915, which are arranged outside the shielding element 910, and, furthermore, two ultrasonic lines 920, which are arranged inside the shielding element 910.

FIG. 10 shows a flowchart of an exemplary embodiment of a method 1000 for producing a line device in accordance with one exemplary embodiment. In accordance with one exemplary embodiment, the method 1000 is carried out and/or triggered on a device 1010 for producing a line device.

In a step 1020, a guide cannula and an electrical conducting element are provided. In a step 1030 of the method 1000, the electrical conducting element is arranged inside the guide cannula, in order to produce a line device.

If an exemplary embodiment comprises an “and/or” conjunction between a first feature and a second feature, then such a conjunction should be understood to mean that the exemplary embodiment comprises both the first feature and the second feature in accordance with one embodiment and comprises either just the first feature or just the second feature in accordance with another embodiment.

1-15. (canceled)

16. A conduit for a cardiac assist system, the conduit comprising:
a guide cannula comprising a sheath; and
an electrical conducting element attached to the sheath of the guide cannula, wherein the electrical conducting element comprises a plurality of layers and a sensor contact region configured to contact at least one sensor.

17. The conduit of claim 16, wherein the plurality of layers comprises a conductive layer, wherein the conductive layer is made at least partially of a gold material.

18. The conduit of claim 16, wherein the plurality of layers comprises an insulating layer, wherein the insulating layer is made at least partially of a polyimide material.

19. The conduit of claim 16, wherein the electrical conducting element comprises a shielding element.

20. The conduit of claim 19, wherein the shielding element comprises conductive layers.

21. The conduit of claim 20, wherein the shielding element comprises a through-contact between the conductive layers.

22. The conduit of claim 19, wherein the electrical conducting element comprises a plurality of lines, wherein at least one line of the plurality of lines is arranged inside a layer of the plurality of layers, and wherein at least one line of the plurality of lines is arranged outside the shielding element.

23. The conduit of claim 16, wherein the electrical conducting element comprises a signal generator contacting region configured to contact at least one signal generator.

24. The conduit of claim 16, wherein the sensor contact region is configured to receive at least two sensors, wherein the sensor contact region is rectangular in shape and comprises at least two edges, and wherein the sensor contact region is bent at the at least two edges.

25. The conduit of claim 23, wherein the signal generator contacting region comprises at least two bent contact points.

26. The conduit of claim 16, wherein the electrical conducting element comprises a connection point element, wherein the connection point element comprises a plurality of connecting points arranged circumferentially on an outer edge the connection point element.

27. The conduit of claim 16, wherein the sheath is cylindrical and comprises a spiral structure, and wherein the sheath of the guide cannula comprises a metal-containing alloy.

28. The conduit of claim 16, wherein each layer of the plurality of layers of the electrical conducting element has a thickness ranging between 5 μm and 15 μm; and wherein the electrical conducting element is formed in a meandering manner.

29. A cardiac assist system comprising:
a conduit arranged between a sensor head unit and an end unit;
a first connecting element arranged between the conduit and the sensor head unit; and
a second connecting element arranged between the conduit and the end unit.

30. The cardiac assist system of claim 29, wherein the conduit further comprises:
a guide cannula comprising a sheath having a spiral structure; and
an electrical conducting element coupled with the spiral structure of the sheath of the guide cannula, the electrical conducting element comprising a plurality of layers and a sensor contact region configured to contact at least one sensor.

31. A method for producing a conduit for a cardiac assist system, the method comprising:
providing a guide cannula comprising a sheath; and
applying an electrical conducting element on at least a portion of the sheath of the guide cannula, the electrical conducting element comprising a sensor contact region configured to contact at least one sensor.

32. The method of claim 31, wherein the sheath is cylindrical and comprises a spiral structure, and wherein the electrical conducting element is applied on at least a portion of the spiral structure of the sheath.

33. A system for producing a conduit for a cardiac assist system, the system comprising:
a machine-readable storage device storing therein computer-readable instructions; and
a processor, wherein the computer-readable instructions, when executed by the processor, cause the processor to perform steps of:
applying an electrical conducting element on at least a portion of a sheath of a guide cannula, the electrical element comprising a sensor contact region for contacting at least one sensor.

34. The system of claim 33, wherein the sheath is cylindrical and comprises a spiral structure, and wherein the electrical conducting element is applied on at least a portion of the spiral structure of the sheath.

35. A non-transitory computer storage device stored thereon computer-readable instructions that, when executed by a processor of a manufacturing device, cause the processor to perform steps of:
applying an electrical conducting element on at least a portion of a sheath of a guide cannula, the electrical element comprising a sensor contact region for contacting at least one sensor.
